SEQUENCE LISTING (3 sequences)

SEQ ID NO: 1 - DNA, 20 bp, Artificial Sequence
  Primer - sense
gtgtctgcggcgttttatca

SEQ ID NO: 2 - DNA, 21 bp, Artificial Sequence
  Primer - antisense
gacaaacgggcaacatacctt

SEQ ID NO: 3 - DNA, 28 bp, Artificial Sequence
  HBV Probe
cctctkcatcctgctgctatgcctcatc

(19) United States
(12) Patent Application Publication
(10) Pub. No.: US 2023/0228736 A1
(43) Pub. Date: Jul. 20, 2023

(54) IN VITRO HBV CORE PROTEIN ASSAY

(71) Applicant: Janssen Sciences Ireland Unlimited Company, Co Cork (IE)

(72) Inventors: Jan Martin BERKE, Beerse (BE); Sarah SAUVILLER, Geel (BE); Karen Maya VERGAUWEN, Edegem (BE); Steffen JAENSCH, Antwerpen (BE); Emmanuel Marie Paul Ernest GUSTIN, Sint-Katelijne-Waver (BE)

(21) Appl. No.: 17/492,238

(22) Filed: Oct. 1, 2021

Related U.S. Application Data
(60) Provisional application No. 63/086,734, filed on Oct. 2, 2020.

(51) Int. Cl. G01N 33/50; G01N 33/576; C07K 14/02

(52) U.S. Cl. CPC G01N 33/5008; G01N 33/5761; C07K 14/02; G01N 2500/02

(57) ABSTRACT

Provided herein is a method of identifying a compound that induces the formation of Hepatitis B Virus (HBV) aberrant viral capsid structures.

### CROSS-REFERENCE TO RELATED APPLICATIONS

This application claims priority to U.S. Patent Provisional Application No. 63/086,734. filed on Oct. 2, 2020, which is incorporated here in its entirety.

### REFERENCE TO SEQUENCE LISTING SUBMITTED ELECTRONICALLY

This application contains a sequence listing, which is submitted electronically via EFS-Web as an ASCII formatted sequence listing with a file name of “065814-123US2_sequence_listing_ST25.txt” and a creation date of Nov. 21, 2022, and having a size of 816 bytes. The sequence listing submitted via EFS-Web is part of the specification and is herein incorporated by reference in its entirety

### BACKGROUND

Worldwide, approximately 2 billion people have been infected with hepatitis B virus (HBV), of which currently over 250 million people have developed a chronic HBV infection. Progression to cirrhosis or the development of hepatocellular carcinoma are the main complications of HBV with a mortality rate of 887,000 people a year. With a limited number of patients treated today (only 4-5% receive treatment in the US), two treatment approaches are available for chronic HBV infection. One option is to treat patients life-long with well-tolerated nucleos(t)ide analogues to suppress HBV replication. Another option is to treat patients with pegylated interferon-α for 48 weeks, a treatment being associated with significant side effects. Functional cure for HBV is defined as loss of hepatitis B surface antigen (HBsAg) with or without HBsAg seroconversion and with undetectable HBV DNA serum levels. Currently, only 5-10% of treated patients achieve functional cure. Accordingly, new HBV anti-viral compounds should have a new mechanism of action, and, alone or in combination, should be well tolerated by patients and increase functional cure rates.

HBV core protein has multiple functions in the viral life cycle and is an attractive target for new anti-viral therapies. Capsid assembly modulators (CAMs) target the core protein and induce the formation of either morphologically normal (CAM-N) or aberrant structures (CAM-A), both devoid of genomic material. To date a diverse family of CAM-N chemotypes has been identified, but in contrast, described CAM-As are based on the heteroaryldihydropyrimidine (HAP) scaffold only. Accordingly, there exists a need to identify novel CAMs that induce the CAM-A HBV phenotype. Provided herein is a robust cellular assay for the accurate and rapid identification of compounds that induce the CAM-A HBV phenotype.

### SUMMARY

In one aspect, the disclosure provides a method of identifying a compound that induces the formation of Hepatitis B Virus (HBV) aberrant viral capsid structures, the method comprising the steps of:

a) providing a plurality of HBV-replicating cells;
b) contacting the HBV-replicating cells with at least one test compound;
c) fixating the HBV cells of step b);
d) incubating the fixated HBV cells of step c) with an antibody that specifically binds to HBV core protein, thereby labelling HBV core protein fixated HBV cells;
e) imaging the HBV core protein-labeled fixated HBV cells; and
f) determining the presence of an HBV core protein speckle phenotype,
wherein the presence of an HBV core protein speckle phenotype indicates that the test compound induces the formation of HBV aberrant viral capsid structures.

### BRIEF DESCRIPTION OF THE DRAWINGS

FIG. 1 presents results related to the analysis of HepG2.117 cell expression of HBV-core proteins. FIG. 1 panel A shows confocal images of HepG2.117 cells with or without the expression of HBV-core proteins in the absence (−DOX) or presence of doxycycline (+DOX), respectively. The cells were immunostained with Hoechst (cyan) and CellMask™ DeepRed (purple) for for nuclear staining and cellular demarcation respectively. FIG. 1 panel B shows HepG2.117 cells immunolabeled with primary core antibodies, either polyclonal rabbit antibody from Dako or monoclonal mouse antibody from Abcam which were detected with Alexa 488 secondary antibodies and counterstained with Hoechst and CellMask™ DeepRed. Fluorescence was detected by confocal imaging. The cellular core mean fluorescence intensity levels are plotted for each primary antibody for core expressing and non-expressing HepG2.117 cells.

FIG. 2A shows distinct core immunostaining phenotypes of HepG2.117 cells when treated with CAM-N or CAM-A compounds. HepG2.117 cells were cultured without (A) or with HBV core expression (B, C, D) and treated with DMSO as a control (A, B), a CAM-N(C) or a CAM-A (D) compound. Cells were immunostained for HBV core (green) and co-stained with Hoechst (cyan) and CellMask™ Deep Red (purple). Various core texture-related features were analyzed with a z-score normalization relative to non-treated core-expressing HepG2.117 cells to identify the best feature that represents the “aggregated core” phenotype of the compound in a dose-dependent way, BAY41-4109 shown in (D) and JNJ-61030827 shown in (C).

Cells were cultured without (A) or with induction of HBV core expression (B, C, D) and treated with vehicle (DMSO) (A, B), a CAM-N compound (C) or a CAM-A compound (D). Cells were immunostained for HBV core (green) and co-stained with Hoechst (cyan) and CellMask™ DeepRed (purple). The various texture-related features in the core-expressing, treated cells were analyzed with a z-score normalization relative to non-treated controls to identify the “core aggregated” phenotype as the best representative feature induced by the compounds in a dose-dependent manner.

FIG. 2B shows representative images of the core aggregated phenotype in cells treated with BAY41-4109 shown in (E) and JNJ-61030827 shown in (F).

FIG. 3 shows further quantification of the CAM-A dose-dependent core speckling phenotype of core-expressing HepG2.117 cells treated with 3 internal HAP scaffolds and immunolabeled to visualize the core proteins, nuclei and cell demarcation. The cells were analyzed with a z-score normalization relative to non-treated core-expressing HepG2.117 cells

FIG. 4A shows time kinetic of aggregated core induction and exclusion of toxicity-induced core aggregation. HepG2.117 cells were treated with a diverse set of CAM-A and CAM-N scaffolds for 48 hours, 72 hours, and 96 hours. After immunostaining for core, cytosolic and nuclear core aggregation was plotted for the tested concentration range. Control results of HepG2.117 cells lacking core expression are plotted aside the x-axis dose-response as background controls (BC), while results of core-expressing cells are plotted aside the x-axis as the low control value (LC). The compound-induced cell toxicity was measured by monitoring the overall cell count that was imaged in each well. All raw data is normalized as a percentage compared to the core expressing HepG2.117 cells (LC) (panel A).

To exclude that compound-induced cell toxicity leads to the formation of aberrant core structures, a panel of diverse known toxicity reference compounds with different mechanism of actions was tested in parallel.

FIG. 4B depicts two representative results showing that although toxicity was induced, no cytosolic or nuclear aggregated core was observed in the cells (panel B).

FIG. 5A shows the plate layout for an inter and intra-assay variability of the high-content imaging aggregation assay. 16 known CAM-A and CAM-N compounds in 11-dose range concentrations were tested in duplicates. For an inter-assay variability check, the compounds were tested in three independent experiments. For intra-assay variability assessment, two plates were tested in parallel within the same experiment group. For a combined inter and intra-assay variability check, HepG2.117 cells with different passage numbers were used while one plate group was processed by a different operator to assess operator-dependent variability.

FIG. 5B and FIG. 5C show results of a selection of the 16 CAM-A and CAM-N compounds that were tested for their aggregating capability. Dose dependent cytosolic (FIG. 5B) and nuclear specking (FIG. 5C) was observed for 8 compounds.

FIG. 6A shows the reproducibility of the high-content imaging aggregation assay. Logistic regression curve fitting graphs of published CAM molecules that were among the 16 compounds tested for their aggregation capability are shown (Panel A).

FIG. 6B shows the robustness of the assay which is reflected by the narrow range between minimum and maximum calculated EC₅₀ values between the different plate groups for the 8 compounds that induced core aggregation. In FIG. 6B, the symbols have the following meaning: ● INTER-variability A; ▪ INTER-variability B; ▴ INTRA-variability A; ▾ INTRA-variability b; ♦ INTRA-variability C; ● NO blocking in immunostaining procedure.

FIG. 7 shows the effectiveness of the high-content imaging assay for screening purposes through the calculation of the assay's Z′ factor in a subset of core-expressing cells.

FIG. 8 shows a schematic for the screening approach taken to identify novel chemotypes that induce a CAM-A phenotypic cell morphology.

FIG. 9 shows the properties of Compound A, a novel non-HAP CAM-A scaffold able to induce cytosolic and nuclear HBV core aggregation identified from a high-throughput screen of 700,000 compounds.

FIG. 10 shows capsid depletion assay (Panel A) and electron microscopy confirmation (Panel B) of Compound A CAM-A phenotype and mode of action.

### DETAILED DESCRIPTION

Described herein are methods of identifying a compound that induces the formation of Hepatitis B Virus (HBV) aberrant viral capsid structures. Compounds that induce the formation of HBV aberrant viral capsid structures may be identified by determining the presence of an HBV aggregated core protein phenotype in a cellular immunofluorescent HBV core protein assay. The methods described herein provide a reliable and fast way to determine if a compound induces the formation of HBV aberrant viral capsid structures, an important step in the development of HBV therapies.

### Definitions

As used herein, the terms “Hepatitis B Virus viral capsid” or “HBV viral capsid” or “HBV capsid” refers to the proteinaceous core of HBV that contains the viral genomic material. The capsid is composed of multiple copies of the HBV core protein, which assemble to form the icosahedral capsid.

As used herein, the terms “HBV core protein,” “HBc,” or “Cp” refers to the HBV capsid protein monomers. HBV capsid assembly occurs when two α-helical HBc monomers form a dimer via an intradimer interface, and numerous dimers subsequently form the icosahedral capsid via a dimer-dimer interface. The HBV capsid assembles around HBV pregenomic RNA (pgRNA) and the HBV reverse transcriptase (the P protein).

As used herein, the term “aberrant viral capsid structure” refers to an HBV capsid structure with a morphology that differs from the wild-type capsid structure morphology. An aberrant viral capsid structure includes, but is not limited to, a capsid that is larger than a wild-type, or functional, HBV capsid, a capsid that forms aggregates, or both.

### Capsid Assembly Modulators

As used herein, the term “capsid assembly modifier,” or “capsid assembly modulator,” or “CAM,” or “core protein allosteric modulator,” or “CpAM” is a compound that targets the HBV core protein and induces the formation of either morphologically normal (“CAM-N”) or aberrant structures (“CAM-A”). Both CAM-N and CAM-A structures lack packaged genomic material, i.e., pgRNA or double-stranded DNA. CAMs interfere with HBV replication via multiple pathways. First, they are able to accelerate the kinetics of capsid assembly and thus prevent encapsulation of the HBV polymerase-pregenomic RNA (pol-pgRNA) complex, which impairs the production of infectious virions. Secondly, CAMs interfere with covalently closed circular DNA (cccDNA) transcription or prevent the de novo formation of cccDNA in the early steps of HBV infection. Third, a depleted pool of core proteins can upregulate the transcription of interferon and interferon-stimulated genes, thereby restoring a host's innate immune response.

CAMs that induce CAM-N structures include, but are not limited to, phenylpropenamide and derivatives thereof, and sulfamoylbenzamide (“SBA”) and derivatives thereof. Non-limiting examples of phenylpropenamide derivatives include AT-61 and AT-130 (Delaney I V et al. Antimicrob Agents Chemother. 46(9): 3057-3060. 2002). Non-limiting examples of sulfamoylbenzamide derivatives include JNJ-632, JNJ-827, and JNJ-6379 (Berke et al. Antimicrob Agents Chemother. 61(8). 2007; Lahlali et al. Antimicrob Agents Chemother. 62(10). 2018; Berke et al. Antimicrob Agents Chemother. DOI: 10.1128/AAC.02439-19. 2020; Vandyck et al. J Med Chem. 61(14): 6247-6260. 2018).

CAMs that induce CAM-A structures include, but are not limited to, heteroaryldihydropyrimidine (“HAP”) and derivatives thereof. Non-limiting examples of HAP derivatives include BAY41-4109, HAP-1, HAP-12, GLS-4 (i.e., morphothiadin or 6[R,S]-ethyl-6-(2-bromo-4-fluorophenyl)-4-(morpholinomethyl)-2-(thiazol-2-yl)-1,6-dihydropyrimidine-5-carboxylate mesylate), and JNJ-890 (Lahlali, supra; Boucle et al. Bioorg Med Chem Lett. 27(4): 904-910. 2017; Deres et al. Science. 299(5608): 893-6. 2003; Ren et al. Bioorg Med Chem. 25(3): 1042-1056. 2017; Stray et al. Proc Natl Acad Sci USA. 102(23): 8138-43. 2005). Additional CAMs can be selected for example from WO2020169784.

### HBV-Replicating Cells

As used herein, the term “HBV-replicating cell,” or “HBV-producing cell,” or “HBV-expressing cell,” refers to a cell line that has been modified to produce HBV viral particles. In some embodiments, HBV viral particles comprise a wild-type, normal morphology capsid that contains the HBV genetic material. In some embodiments, HBV viral particles comprise a wild-type, normal morphology capsid that lacks the HBV genetic material (i.e., empty capsids, CAM-N structures). In some embodiments, HBV viral particles comprise an aberrant morphology capsid that lacks the HBV genetic material (i.e., large, aggregated capsids, CAM-A structures).

In some embodiments, the HBV-replicating cell is transiently transfected with an expression vector containing the HBV genome. In some embodiments, the HBV-replicating cell is stably transfected with an expression vector containing the HBV genome. The expression vector containing the HBV genome can express the HBV pregenomic RNA (pgRNA), which encodes for the HBV polymerase and core protein. In some embodiments, HBV pgRNA expression is under the control of a constitutive promoter. In some embodiments, HBV pgRNA expression is under the control of an inducible promoter. In some embodiments, the inducible promoter is a tetracycline (Tet)-inducible promoter. The Tet-inducible promoter is regulated by the presence of doxycycline (Dox). When Dox is present in the cell culture media, the Tet promoter is repressed and the HBV pgRNA is not expressed. When Dox is removed from the cell culture media, the Tet promoter is activated and the HBV pgRNA is expressed.

In some embodiments, the HBV-replicating cell is a liver-derived cell. Non-limiting examples of liver-derived cells include HepG2, HepaRG, Huh7, primary hepatocytes, and Tupaia belangeri hepatocytes. In some embodiments, the HepG2, HepaRG, Huh7, primary hepatocyte, and Tupaia belangeri hepatocyte cells have been previously transfected or infected with HBV virus. Methods of transfection and infection are known to the skilled person. In some embodiments the liver-derived HepG2 cells comprise HepG2.117, HepG2.2.15 and HepAD38 cells. In some embodiments, the liver-derived cell comprises the HepG2.117 cell. The HepG2.117 cell is stably transfected with the HBV genome under the control of the Tet-inducible promoter. The HepG2.117 is described in more detail in Sun et al. (J. Hepatol. 45(5):636-45. 2006).

The method of the disclosure employs a plurality of HBV-replicating cells. The plurality of cells can be incubated in the wells of a multi-well tissue culture plate, such as a 6-well, 24-well, 48-well, 96-well, or 384-well tissue culture plate. In some embodiments, the tissue culture plate comprises an optically clear bottom to facilitate imaging of expressed HBV core protein.

### HBV Aggregated Core Protein Phenotype

As used herein, the term “HBV aggregated core protein phenotype” refers the appearance of HBV core proteins within HBV-replicating cells. The aggregated phenotype is characterized, in part, by the presence of discrete dots or spots within one or both of the nucleus and cytoplasm of the cells. The aggregated core structures can appear as a dot-like pattern within one or both of the nucleus and cytoplasm of the cells. The aggregated core structures are visualized with a HBV core protein imaging reagent, such as an anti-HBV core protein antibody in combination with a fluorescently labeled secondary antibody.

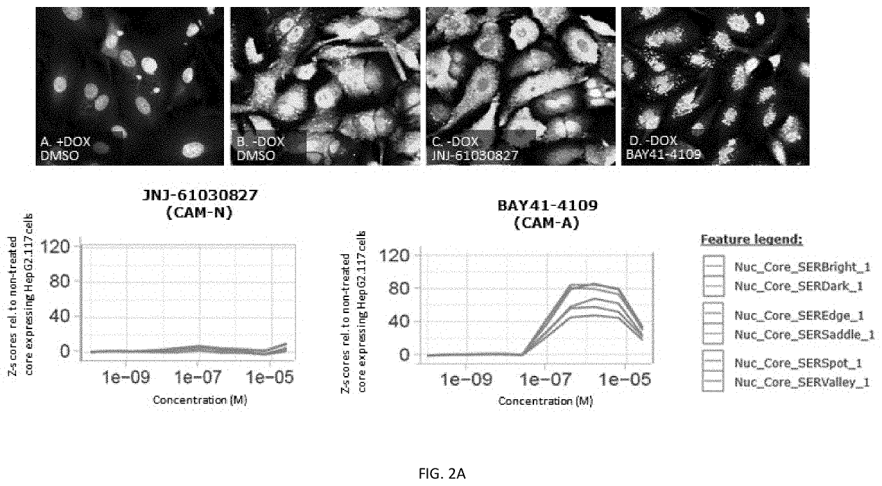

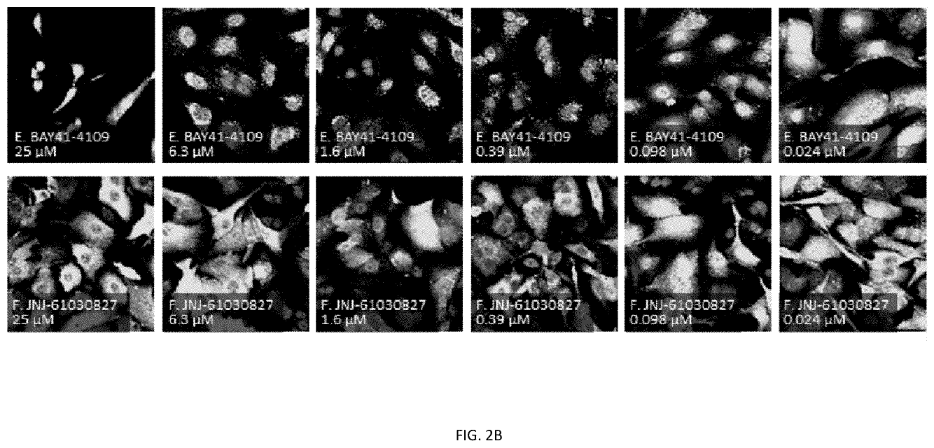

A representative image of the HBV aggregated core protein phenotype is shown in FIG. 2A, in the fluorescent image labeled “D. −DOX BAY41-4109.” BAY41-4109 is a CAM that induces the CAM-A structures described above, and is the type of compound that should be identified in the methods described herein. This representative HBV aggregated core proteinphenotype image can be compared against the fluorescent image labeled “C. −DOX JNJ-61030827” in FIG. 2A. JNJ-61030827 is a CAM that induces the CAM-N structures described above. In some embodiments, a compound that does not induce the formation of HBV aberrant viral capsid structures produces an HBV core protein uniform imaging phenotype, such as the one shown in the fluorescent image labeled “C. −DOX JNJ-61030827” in FIG. 2A.

As used herein, the term “uniform imaging phenotype” refers to a cellular image wherein the detection signal (e.g., fluorescent signal) is diffuse throughout the cell (i.e., in the cytoplasm and nucleus), and there is a minimal amount of discrete spots or dots from the detection signal. In some embodiments, the HBV core protein uniform imaging phenotype comprises an even distribution of HBV core protein in the nucleus and cytoplasm of the HBV-replicating cells

### Test Compounds & HBV-Replicating Cell Imaging

As used herein, the term “test compound” refers to a compound that is suspected of having, or being investigated for having, CAM properties, as described above. In the method of the disclosure, at least one test compound is contacted with the HBV-replicating cell or plurality of HBV-replicating cells. In some embodiments, the test compound is incubated or contacted with the plurality of HBV-replicating cells prior to inducing HBV expression. In some embodiments, the test compound is incubated or contacted with the plurality of HBV-replicating cells simultaneously with inducing HBV expression. In some embodiments, the test compound is incubated or contacted with the plurality of HBV-replicating cells after inducing HBV expression.

In some embodiments, the test compound is provided at a fixed dose or at a dose range (i.e., each well with a plurality of cells receives a fixed dose, with multiple wells used to test a range of doses). In some embodiments, the fixed dose is about 0.1 nM, about 1 nM, about 10 nM, about 100 nM, about 1 μM, about 10 μM, about 20 μM, about 25 μM, about 30 μM, about 40 μM, about 50 μM, about 60 μM, about 70 μM, about 80 μM, about 90 μM, or about 100 μM. In some embodiments, the dose range is about 0.1 nM to about 100 μM.

In some embodiments, the at least one test compound is a HAP derivative.

In some embodiments, the at least one test compound is a non-HAP derivative.

In some embodiments, the at least one test compound is incubated with the plurality of HBV-replicating cells for a time sufficient to observe phenotypic changes in HBV capsid morphology. In some embodiments, the at least one test compound is incubated with the plurality of HBV-replicating cells for about 12 hours, about 24 hours, about 36 hours, about 48 hours, about 60 hours, about 72 hours, about 84 hours, about 96 hours, about 108 hours, or about 120 hours. In some embodiments, the at least one test compound is incubated with the plurality of HBV-replicating cells for about 72 hours.

Following test compound incubation with the plurality of HBV-replicating cells, the plurality of HBV-replicating cells are then fixed and permeabilized. Fixation can be performed by incubating the cells with an appropriate fixation reagent. In some embodiments, the plurality of HBV-replicating cells are fixed with formaldehyde. Following fixation, the plurality of HBV-replicating cells can be permeabilized to facilitate staining of cellular structures and labeling of HBV core proteins (e.g., labeling with an anti-HBV core protein antibody comprising a fluorescent label or a fluorescently labeled secondary antibody with binding specificity for the anti-HBV core protein antibody). In some embodiments, the plurality of HBV-replicating cells are permeabilized with a surfactant. In some embodiments, the plurality of HBV-replicating cells are permeabilized with Triton X-100.

As used herein, the term “HBV core protein-labeled HBV-replicating cells” refers to HBV-replicating cells that have been incubated with one or more imaging reagents that permit the visualization of expressed HBV core proteins in the HBV-replicating cells. In some embodiments, the HBV-replicating cells (e.g., the plurality of HBV-replicating cells) are incubated with the one or more imaging reagents for an amount of time sufficient to achieve an imaging signal (e.g., fluorescent intensity signal) that is higher that a background imaging signal (i.e., HBV-replicating cells that have not been incubated with the one or more imaging reagents). In some embodiments, the imaging reagent is an antibody that specifically binds to HBV core protein. In some embodiments, the antibody that specifically binds to HBV core protein is polyclonal or monoclonal. In some embodiments, the antibody is a primary polyclonal rabbit HBV core antibody. In some embodiments, the antibody is a primary monoclonal mouse HBV core antibody, such as the Abcam C1 antibody. Monoclonal antibodies may result in better sensitivity with a better signal to noise ratio. Suitable further antibodies include the DAKO antibody and the Abcam Ab8637 antibody, or equivalent.

In some embodiments, the antibody that specifically binds to HBV core protein is labeled with a detectable moiety (e.g., the primary monoclonal mouse HBV core antibody). In other embodiments, the antibody that specifically binds to HBV core protein is unlabeled. When the anti-HBV core protein antibody is unlabeled, a second antibody (secondary antibody) that is labeled is incubated the HBV-replicating cells following the initial incubation with the anti-HBV core protein antibody. The second antibody comprises binding specificity to the anti-HBV core protein antibody. In some embodiments, the second antibody is a goat anti-rabbit antibody or a goat anti-mouse antibody.

As used herein, the term “antibody” or “antigen binding protein” refers to an immunoglobulin molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)2 fragments, Fab′ fragments, Fv fragments, recombinant IgG (rIgG) fragments, single chain variable fragments (scFv) and single domain antibodies (e.g., sdAb, sdFv, nanobody) fragments. The term “antibody” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof. Moreover, the term “antibody” includes all of the different human isotypes of antibodies, IgA, IgD, IgE, IgG, and IgM.

The antibody of the disclosure (i.e., the anti-HBV core protein antibody or secondary antibody) is labeled with a detectable moiety.

In some embodiments, detectable moiety is a fluorescent label. In some embodiments, a fluorescently labeled detectable moiety is detected and quantified by, for example, measuring the increased fluorescence polarization arising from the complex-bound peptide relative to that of the free peptide.

In some embodiments, detectable moiety is a fluorescent label is fluorescein or a derivative thereof. Non-limiting examples of fluorescein derivatives include Alexa Fluor compounds and fluorescein isothiocyanate (FITC). In some embodiments, the fluorescent label is Alexa Fluor-488. In some embodiments, the Alexa Fluor-488 label is conjugated to goat anti-rabbit or goat anti-mouse secondary antibody with binding specificity to the anti-HBV core protein antibody.

In some embodiments, the antibody is labeled with a detectable moiety selected from the group consisting of fluorescein or a derivative thereof, biotin, copper-DOTA, biotin-PEG3, aminooxyacetate, ¹⁹FB, ¹⁸FB and FITC-PEG3. In other embodiments, the antibody is labeled with the detectable moiety consisting of ⁶⁴Cu DOTA, ⁶⁸Ga DOTA, ¹⁸F, ⁶⁴Cu, ⁶⁸Ga, ⁸⁹Zr, ¹²⁴I, ⁸⁶Y, ^{94m}Tc, ^{110m}In, ¹¹C and ⁷⁶Br.

In some embodiments, the HBV core protein-labeled HBV-replicating cells are further incubated with a nuclear dye and cell demarcation dye. The purpose of the nuclear dye is to provide a delineation between structures, such as HBV core protein structures, in the nucleus of a cell and the cytoplasm of the cell. The purpose of the cell demarcation dye is to provide a delineation between one cell and adjacent cells. In some embodiments, the nuclear dye is a fluorescent dye. In some embodiments, the nuclear dye is DAPI (4′,6-diamidino-2-phenylindole). In some embodiments, the nuclear dye is a Hoechst stain. In some embodiments, the nuclear dye is Hoechst 33258.

In some embodiments, the cell demarcation dye is a CellMask™ fluorescent dye, such as CellMask™ Green Plasma Membrane Stain, CellMask™ Orange Plasma Membrane Stain, or CellMask™ DeepRed Plasma Membrane Stain. In particular, the cell demarcation dye is CellMask™ DeepRed Plasma Membrane Stain.

In some embodiments, the HBV core protein-labeled HBV-replicating cells are imaged. The manner of imaging will depend on the way the HBV core protein was labeled. In some embodiments, the HBV core protein-labeled HBV-replicating cells are immunofluorescently labeled, as described above. The HBV core protein-labeled HBV-replicating cells can be imaged by confocal microscopy. The confocal microscopy can be performed with any known confocal microscope. In some embodiments, the imaging is performed manually. In some embodiments, the imaging is performed automatically (i.e., by a machine).

In some embodiments, the Opera Phenix™ high-content screening system (Perkin Elmer) can be used for automated imaging. Other alternatives include the CV7000 or CV8000 (Yokogawa).

In some embodiments, a 405-nm laser can be used to excite the nuclear dye (e.g., Hoechst 33258, emitting its fluorescent light at 461-nm). A 635-nm laser can be used to excite the cell demarcation dye (e.g., CellMask™ Deep Red, emitting its fluorescent light at 655-nm). A 488-nm laser can be used to excite the labeled HBV core proteins in the HBV core protein-labeled HBV-replicating cells (e.g., Alexa Fluor-488 fluorophores, with a 500/550-nm emission filter). It will be readily apparent to the skilled artisan which wavelength laser is appropriate based on the choice of dye or fluorescent label employed.

Following imaging and image collection, the presence of the HBV aggregated core protein phenotype described above is determined. The presence of the HBV aggregated protein phenotype can be determined following the steps of:

i) quantifying the number and/or image intensity of HBV core protein aggregates in the cell nucleus and cell cytoplasm of individual HBV-replicating cells within the plurality of HBV-replicating cells;
ii) calculating the average number and/or image intensity of HBV core protein aggregates in the cell nucleus and cell cytoplasm, thereby producing an average value; and
iii) comparing the average value of HBV core protein aggregates to an average value of HBV core protein aggregates in HBV-replicating cells that were not incubated with the at least one test compound, wherein a higher average value in HBV-replicating cells incubated with the at least one test compound compared to the HBV-replicating cells that were not incubated with the at least one test compound indicates the presence of the HBV core protein speckle phenotype.

More in particular, the presence of the HBV aggregated protein phenotype can be determined following the steps of:

i) quantifying the levels of HBV core protein by calculating the average core staining intensity of individual HBV-replicating cells
ii) classifying each HBV-replicating cell as core-expressing or core-suppressed by comparing the intensity calculated in i) to an intensity threshold calculated based on core-suppressed cells (“background control”)
iii) quantifying the extent of HBV core protein aggregation in each cell by calculating a set of texture features derived from the Core staining channel; selecting the texture feature that results in the best separation between negative control wells (core-expressing cells but no compound treatment) and positive control wells (core-expressing cells treated with a reference compound known to induce Core protein aggregation); thereby producing an average value of the selected texture feature across all cells or only the cells classified as core-expressing in step ii)
iv) normalizing the selected texture feature such that the median of the negative control wells on the plate corresponds to a value of 0% and the median of the positive control wells on the plate corresponds to a value of 100%.

### EXAMPLES

### Example 1—Materials and Methods

### HepG2.117 Cell Culture

The human hepatoma-derived cell line HepG2.117 was employed as the HBV-replicating cells for this disclosure, and are described in greater detail in Sun et al., supra. The HepG2.117 cells were cultured in Dulbecco's modified Eagle's medium (DMEM; Lonza, Basel, Switzerland) supplemented with 10% fetal calf serum (Biowest, Nuaillé, France), 2 mM alanyl-glutamine (Sigma-Aldrich, St. Louis, Mo.), 200 μg/ml gentamicin (Gibco, Carlsbad, Calif.), 500 μg/ml geneticin (Gibco), 70 μg/ml hygromycin B (Roche, Mannheim, Germany), and 100 ng/ml doxycycline (Clontech Labs, Mountain View, Calif.). Transcription of pgRNA wass induced by withdrawal of doxycycline from the cell culture medium which results in expression of core and polymerase HBV proteins.

### Antibodies and Cellular Counterstains

Primary polyclonal rabbit HBV core antibody was purchased from Dako (Agilent, Santa Clara, Calif.) and used at a dilution of 1:500, while primary monoclonal mouse HBV core antibody (designated “C1”) was purchased from Abcam (Cambridge, United Kingdom) and used at a dilution of 1:1,500. Secondary Alexa Fluor-488 goat anti-rabbit and Alexa Fluor-488 goat anti-mouse antibodies were purchased at Life Technologies (Carlsbad, Calif.) and used at a dilution of 1:1,000. Hoechst 33258 for identification of nuclei and HCS CellMask™ Deep Red Stain for staining of the entire cell were also purchased at Life Technologies and were both used at a dilution of 1:5,000.

### Reference Compounds

All compounds used in the disclosure were either synthesized in house or at Wuxi AppTech, Shangai, China and had a purity of >95%. GLS-4 was used as the positive high control for the induction of core aggregation. A Panel of 11 known toxicity compounds was used for exclusion of toxicity-induced core aggregation, as shown below in Table 1.

TABLE 1
Panel of 11 known toxicity reference compounds. The different toxicity compounds
are indicated by their compound names and by their mechanism of action.

| Compound | |
|---|---|
| name | Toxicity pathway |
| PFI-1 | highly selective bromo and extra terminal (BET) inhibitor for |
| | bromodomain protein 4 and 2 (BRD4 and BRD2) |
| 5-Fluorouracil | inhibitor of thymidylate synthetase and incorporation into RNA |
| Gemcitabine | nucleic acid synthesis inhibitor |
| R306465 | potent inhibitor of class I histone deacetylases |
| rapamycin | specific mammalian target of rapamycin (mTOR) inhibitor |
| chloroquin | DNA replication and RNA synthesis inhibitor, inhibitor of the activity |
| | of vacuolar phospholipase, vacuolar proteases, and heme polymerase |
| tunicamycin | inducer of endoplasmic reticulum stress in cells by inhibiting first |
| | step in biosynthesis of N-linked glycans in proteins resulting in |
| | many misfolded proteins |
| brefeldin | ATPase inhibitor for protein transport - Golgi apparatus |
| Geldanamycin | natural heat shock protein 90 (HSP90) inhibitor |
| Pevonedistat | small molecule inhibitor of Nedd8 activating enzyme (NAE) |
| sorafenib | multikinase inhibitor of rapidly accelerated fibrosarcoma (Raf-1, |
| | B-Raf), and vascular endothelial growth factor (VEGFR-2) |

### Cellular HBV Core High-Content Imaging Assay

After trypsinization, HepG2.117 cells were washed once with phosphate buffered saline (PBS). Cells were centrifuged at 1,500 rpm for 5 minutes and divided in two fractions. The fraction that was used for the core-suppressed HepG2.117 cell controls (background control, BC) was resuspended in DMEM supplemented with 2% Tet-system-approved fetal calf serum (FCS; Clontech), 2 mM alanyl-glutamine, lx non-essential amino acids (NEAA; Sigma-Aldrich), and 100 ng/ml doxycycline to suppress core expression. The other fraction was resuspended in DMEM supplemented with 2% Tet-system-approved FCS, 2 mM alanyl-glutamine, and 1×NEAA and used for sample wells, the core-expressing low control wells (LC) and aggregated core-expressing high control wells (HC). 2,000 cells per well were seeded for the assay validation, while 1,500 cells per well were seeded in the cellular screening campaign. Test compound-containing 384-well black poly-D-lysine CellCarrier tissue culture treated plates with an optically clear bottom (Perkin Elmer, Waltham, Mass.) were used. Test compounds were used in dose range in the presence of 1% dimethyl sulfoxide (DMSO). Aggregated core expressing high control wells were treated with 1 μM GLS-4. Compound plates with seeded cells were incubated for 72 h at 37° C. and 5% CO₂.

### Z′ Value Determination

HepG2. cells were harvested and fractions for background controls, low controls and high controls were resuspended in assay medium as described above and used on the same 384-well plate. 2,000 cells per well were seeded with 160 wells dedicated for core-positive HepG2.117 cells, 160 wells dedicated for high controls and 64 wells dedicated for background controls. High control wells were treated with 1 μM GLS-4 as a high dose. For a given core texture feature, Z′ values were calculated with the following formula: z′=1-((3× standard deviation of the high controls+3× standard deviation of the low controls)/(| mean of the high controls −mean of the low controls)|). First the Z′ values were calculated based on core texture features averaged across all cells in a well, while followed by a re-analysis that focused on the subset of core expressing HepG2.117 cells. A Z′ value between 0.5 and 1 represents an effective screening assay. Z′ value determination is described in more detail in Zhang et al. (J Biomol Screen, 1999. 4(2): p. 67-73).

### Biochemical Fluorescent Quenching Assay

The modified recombinant HBV core protein Cp*150 (Zlotnick 1997, supra) was made according to Zlotnick et al. (Zlotnick 1996, supra). Following desalting of Cp*150 proteins, the C-terminal residue of the Cp*150 protein was labeled with maleimidyl BoDIPY-FL, and the unreacted dye was removed. A fixed concentration of 30 μM compound or, for profiling, an 11-point serial dilution was added to 384-well plates followed by addition of labeled Cp*150 proteins to all wells. Full assembly and no assembly controls were incubated with NaCl (150 mM for test compounds and high controls and 1 M for the low controls) and 50 mM 4-(2-hydroxyethyl)-1-piperazineethanesulfonic acid (HEPES), respectively. Assembly was initiated in all compound-containing wells with 150 mM NaCl and fluorescent signal was measured 24-hours later (excitation: 485 nm; emission: 538 nm; Envision; Perkin Elmer).

### Immunofluorescence and Confocal Imaging

After about 72 hours, cells were fixed with 5% ultra-pure methanol-free formaldehyde (Polysciences, Warrington, Pa.) for 15 minutes and washed three times with PBS. Next, cells were permeabilized for 30 minutes using 0.5% Triton-X-100 (Sigma-Aldrich) dissolved in PBS and washed again three times with PBS. Cells were incubated for 1 hour with PBS containing 5% bovine serum albumin to block Fc receptors. Core protein was detected with primary monoclonal mouse core antibody at a concentration of 666 ng/ml or polyclonal rabbit core antibody at a concentration of 65 μg/ml in PBS containing 5% bovine serum albumin (Sigma-Aldrich). After overnight incubation at 4° C., cells were washed three times with PBS and primary antibody was detected with 2 μg/ml Alexa Fluor-488 goat anti-mouse or goat anti-rabbit secondary antibody. Nuclei and cytoplasm were co-stained using 2 μg/ml Hoechst 33258 (nuclei) and 500 ng/ml HCS CellMask™ Deep Red (entire cells) solution. After 1 hour of incubation at room temperature, cells were washed three times with PBS.

The Opera Phenix™ high-content screening system (Perkin Elmer) was used for automated imaging. A 405-nm laser with 100% power was used with an exposure time of 200 ms to excite Hoechst 33258 (emitting its fluorescent light at 461-nm); emission light was captured using a 435/480 nm emission filter. Simultaneously, HCS CellMask™ Deep Red signal (emitting its fluorescent light at 655-nm) was excited by a 635-nm laser with a 25 ms exposure time and a laser power reduced to 25%; red emitted light was captured using an emission filter of 650/760 nm. Alexa Fluor 488 fluorophores were excited using a 488-nm laser with 100% power and an exposure of 200 ms, whereby the emission filter 500/550 nm was chosen. This detection was performed independently from Hoechst and HCS CellMask™ Deep Red as the fluorescence spectrum of Hoechst overlaps with that of Alexa Fluor-488. A 20× water immersion objective with a numerical aperture of 1.0 was used for imaging of four fields per well.

### Image Analysis

Images were analyzed with a custom Acapella (Perkin Elmer) script. Nuclei identification was based on the Hoechst 33258 channel, cytoplasm segmentation was based on the HCS CellMask™ Deep Red channel and core protein intensity and texture features were calculated using the Alexa Fluor 488 channel. The cell count feature was used as a measure for cytotoxicity. A reduction in cell count reflects compound induced cytotoxicity. The texture feature “SERSpot” at scale 1.0 was calculated in the nuclear and cytoplasm region of each cell to quantify aggregated core and averaged across all cells on the well-level. The two core texture features were normalized to “% effect” such that the average over low control wells (i.e. non-compound treated core expressing HepG2.117 cells) corresponds to 0% effect and the average over the high controls (i.e. 1 μM GLS-4 treated core-expressing HepG2.117 cells) corresponds to 100% effect. Later on, this analysis was repeated by calculating the average across cells on the well-level of the core texture features only for the core-expressing cells (i.e., cells with above background core intensity) rather than for all cells in the well. To this end, each cell was classified as core expressing or suppressed based on the nuclear core mean intensity feature and comparing it to a threshold calculated based on background control wells (which represent core suppressed cells) as the mean+3 standard deviations across all cells in these background control wells on a given plate.

Non-linear log-logistic curve fitting was chosen for calculation of 50% effective concentration (EC₅₀) values using GraphPad Prism or Phaedra software which is an open source platform for data capture and analysis of high-content screening data (Cornelissen et al. J Biomol Screen, 2012. 17(4): p. 496-506).

### Anti-Viral HBV Activity Assay

HepG2.117 cells were seeded with a cell density of 20,000 cells/well (100 μl) in a 96-well plate in 2% serum containing assay medium as described above. After overnight incubation at 37° C. and 5% CO₂, compound-containing assay medium (100 μl) was added to the cells and these were incubated for an additional 3 days. Supernatant was removed and cells were lysed with 0.33% NP-40 dissolved in water for 5 minutes at 4° C. The plates were vortexed and debris was spun down. Of the lysed supernatant, 35 μl was mixed with 65 μl QuickExtract™ DNA Extraction Solution (Epicentre®) in a 96-well polymerase chain reaction (PCR) plate and incubated at 65° C. for 6 minutes, followed by a 2-minutes heating step at 98° C. and cooled down to 4° C. Of the extracted HBV DNA, 10μ was quantified by a quantitative PCR assay using 15 μl of LightCycler 480 probes master kit mix (Roche) with primers 5′-GTGTCTGCGGCGTTTTATCA-3′ (SEQ ID NO: 1) (sense) and 5′-GACAAACGGGCAACATACCTT-3′ (SEQ ID NO: 2) (antisense) in combination with an HBV probe, 5′-(FAM)-CCTCTKCATCCTGCTGCTATGCCTCATC-3′-(BHQ1) (SEQ ID NO: 3), where FAM is 6-carboxyfluorescein and BHQ1 is Black Hole Quencher 1. The difference in threshold cycle number between compound-treated and untreated wells was used to calculate percent inhibition and calculation of EC₅₀ values. For toxicity analysis, the same HepG2.117 cell density (100 μl) was seeded into compound-containing 96-well plates in 2% serum DMEM assay medium as described above and incubated for 4 days at 37° C. and 5% CO₂. After 4 days, toxicity of compounds was determined using the ATPlite lstep Luminescence Assay System (Perkin Elmer). The selectivity index was calculated as the ratio of the mean 50% cytotoxic concentration (CC₅₀) value over the mean EC₅₀ value in HepG2.117 cells.

Native Polyacrylamide Gel Electrophoresis with Immunoblotting for Detection of Capsids

HepG2.117 cells cultured in 2% serum containing assay medium as described above were plated in a 12-well plate at a cell density of 500,000 cells/well and incubated at 37° C. and 5% CO₂. After overnight incubation, cells were treated with test compound or DMSO for 72 hours at 37° C. and 5% CO₂. Afterwards, cells were washed once with PBS and lysed with 300 μl 10 mM Tris-HCl (pH 7.6), 100 mM NaCl, 1 mM ethylenediaminetetraacetic acid (EDTA), and 0.1% NP-40 supplemented with 1× Halt™ Protease Inhibitor Cocktail (Thermo Fisher Scientific, Waltham, Mass.). After lysis of the cells at 37° C. for 10 minutes, cell debris was discarded by centrifugation at 5,000×g for 10 minutes. Supernatant was collected and protein concentration was determined using the DC (detergent compatible) protein assay (Bio-Rad, Hercules, Calif.). Of protein/sample, 2.2 μg was loaded on a NativePAGE™ Novex® 3-12% Bis-Tris Gel (Invitrogen, Waltham, Mass.) with electrophoresis performed at room temperature using NativePAGE™ Running Buffer and NativePAGE™ Cathode Additive. Proteins were transferred using the Novex® iBlot® Transfer Stack, PVDF, regular size kit followed by a standard immunoblotting procedure using primary polyclonal rabbit core antibody at a concentration of 16.25 μg/ml with overnight incubation. Primary core antibodies were labeled with AIVIDEX™ Goat Anti-Rabbit IgG-Horseradish Peroxidase (GE Healthcare, Chicago, Ill.) and visualized by a Fujifilm (Tokyo, Japan) LAS-3000 imager after using SuperSignal™ West Dura Extended Duration Substrate (Thermo Fisher Scientific).

### Size-Exclusion Chromatography and Electron Microscopy

The assembly domain (amino acids 1 to 149) of recombinant HBV core protein (Cp149) was used in size-exclusion chromatography and electron microscopy studies. Equal volumes of the Cp149 protein and storage buffer were mixed and supplemented with a NaCl solution (final concentration 150 mM). Test compound was added to a final concentration of 20 and the reaction mixture was incubated overnight. Samples were injected into a size-exclusion column (Superdex 200 Increase 10/300 GL; GE Healthcare) and mounted on an Äkta purifier pre-equilibrated with running buffer (50 mM HEPES, 150 mM NaCl, adjusted to pH 7.5) overnight. Samples from the size-exclusion chromatography studies were analyzed by electron microscopy at CODA-CERVA (Brussels, Belgium) to detect HBV capsid particles. The dispersions were negatively stained and analyzed by transmission electron microscopy as described by Mast and Demeestere [38].

### Example 2—Results

Detection of Core-Expressing Cells and Aggregated Core Structures after Treatment with CAM-As

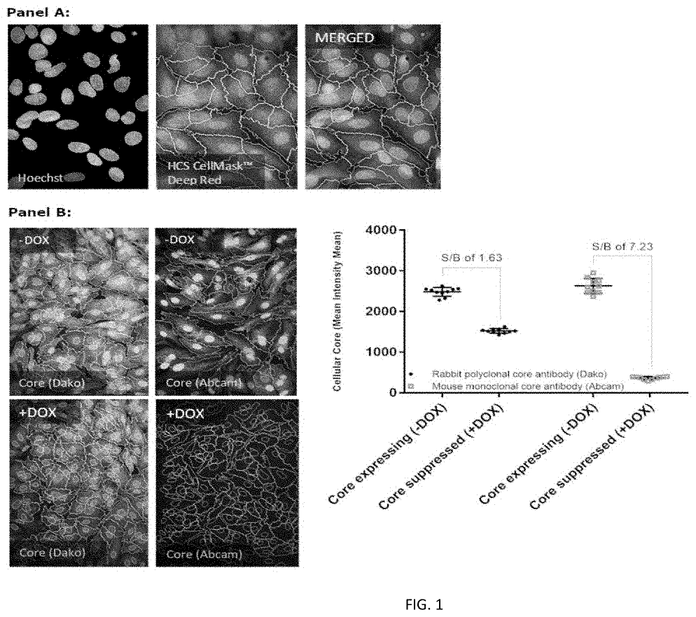

The HepG2.117 cell line was chosen for the development of a high-content image-based cellular assay, where expression of the pgRNA is regulated by a tetracycline (Tet)-responsive minimal cytomegalovirus (CMV) promoter. A simultaneous detection of the nuclei, cytoplasm, and core proteins was possible by combining two cell delineation reagents with a fluorescently labeled antibody staining, where emission of the three fluorescent characteristics are not interfering with each other. Hoechst was used for identification of the individual nuclei of the HepG2.117 cells and HCS CellMask™ Deep Red was used for labeling of the entire cells (FIG. 1, panel A). Core proteins were primarily labeled by Dako polyclonal rabbit core antibody or Abcam monoclonal mouse core antibody that are detected by secondary labeling with Alexa 488 detection antibodies. Cell count was used as a measure of cell viability.

With the absence of doxycycline in the assay medium (“−DOX” in FIG. 1), core expression was detected in the HepG2.117 cells for both the polyclonal rabbit core antibody from Dako and the monoclonal mouse core antibody from Abcam (FIG. 1, panel B). The Dako core staining showed some background staining in core-suppressed HepG2.117 (+DOX) cells resulting in a signal-to-background (S/B) ratio of 1.63 for the overall mean intensity of cellular core. In contrast, using the Abcam core antibody a S/B ratio of 7.23 was obtained due to less background staining of core in core-suppressed HepG2.117 (+DOX) cells, which made it possible to use the core intensity levels of core-suppressed cells to calculate a minimum threshold value to identify core-expressing cells.

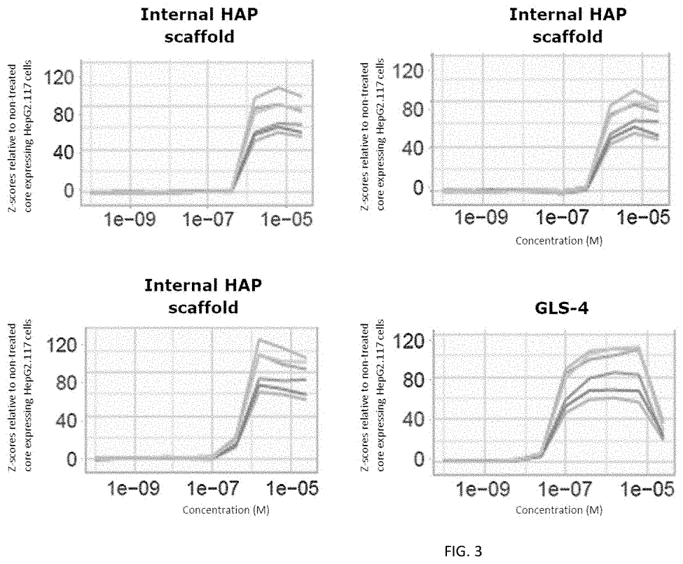

Treatment of HepG2.117 cells with different classes of CAMs resulted in a different phenotypic core immunostaining pattern. HepG2.117 cells treated with a CAM-N compound (JNJ-61030827) showed a uniform core distribution in the nucleus and cytoplasm, while treatment of cells with a CAM-A compound (BAY41-4109) resulted in a more aggregated dot-like pattern of core proteins in the cells (FIG. 2A and FIG. 2B), which are named herein “aggregated corestructures”. Using the Acapella (Perkin Elmer) image analysis scripting language, different texture features (called “SER bright”, “SER dark”, “SER hole”, “SER saddle”, “SER edge”, “SER ridge”, “SER spot” and “SER valley”) that represent different intensity patterns were investigated. Texture features analyze the intensity structure in a defined image region for the occurrence of a typical pattern. The core speckles were recognized by the various texture features which represented a dose-dependent increase of core aggregation for the tested concentration ranges of CAMs. By comparing them to each other, the difference between a CAM-N(JNJ-61030827) and a CAM-A (BAY41-4109) phenotype was best characterized by using “SERSpot” texture feature to quantify the core speckles (FIG. 2A, additional class representatives shown in FIG. 3). These results provided a starting point for further development of a cellular assay that could identify core expression in HepG2.117 cells and for the creation of quantification tools to discriminate between a CAM-N- and a CAM-A-induced phenotype.

### Optimal Time Point for Core Speckling Identification

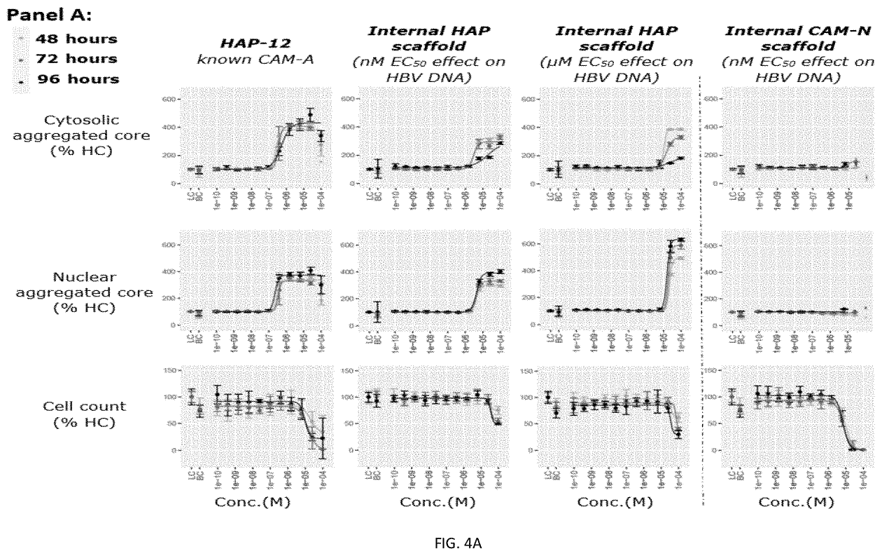

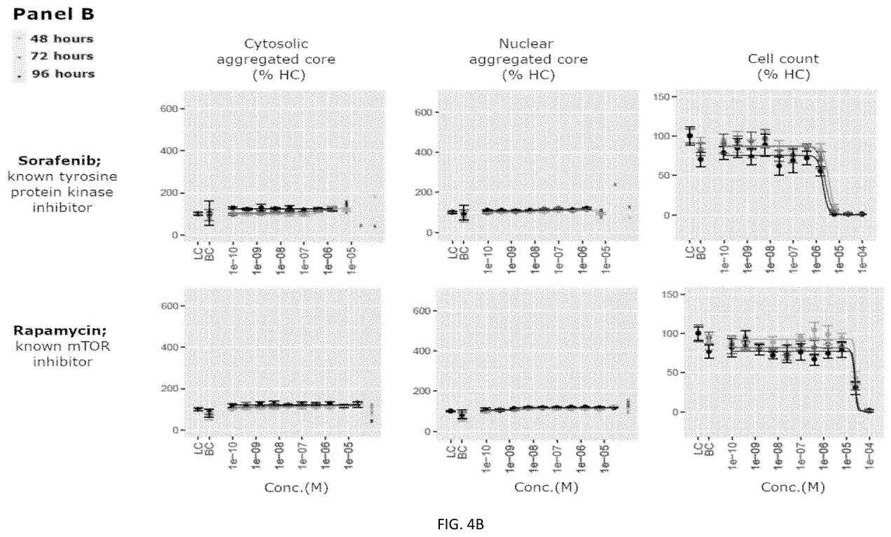

The next step in the assay development was to check if the core aggregation was time dependent. To this end, HepG2.117 cells were treated with a panel of different CAMs (FIG. 4A). The compound panel was tested in triplicate so that the capacity to induce core speckling could be evaluated after 48, 72, or 96 hours of compound treatment. The results showed that at 48 hours, core aggregated both in the cytoplasm and nucleus of HepG2.117 cells. After 72 hours, core aggregation showed a similar level of cytoplasmic core aggregation but a slightly increased nuclear aggregation. In contrast, HepG2.117 cells treated with CAM-As for 96 hours had a pronounced core aggregation localized in the nucleus but had a diminished or different dose-response curve for core aggregation in the cytoplasm. Comparing the three tested incubation times, 72 hours of compound treatment was selected for further assay validation as the cells showed a balanced cytoplasmic and nuclear aggregated core phenotype at this time point. (Representative figures in FIG. 4A). The representative figures also show that the high content imaging assay can identify aggregated core for HAP scaffolds that have a potent HBV anti-viral activity (nM EC₅₀ activity), but if started high enough in concentration, the assay is also able to identify core speckling for HAP scaffolds having less anti-viral activity (μM EC₅₀ activity).

### Exclusion of Cytotoxicity as Possible Cause for Core Aggregation

The concentration range tested for the CAMs showed a dose-dependent effect on the overall cell count. A decrease in cell count would likely represent compound-induced cytotoxicity. To exclude compound-induced cytotoxicity as cause for core aggregation, a panel of toxic compounds with known mechanisms of action was tested (e.g., autophagy, endoplasmic-reticulum-stress related toxicity, or mitochondrial toxicity). All compounds from the cytotoxicity panel showed flat dose-response curves for cytoplasmic and nuclear core speckling, indicating that cytotoxicity does not induce core protein aggregation (Representative figures in FIG. 4B).

### Assay Robustness

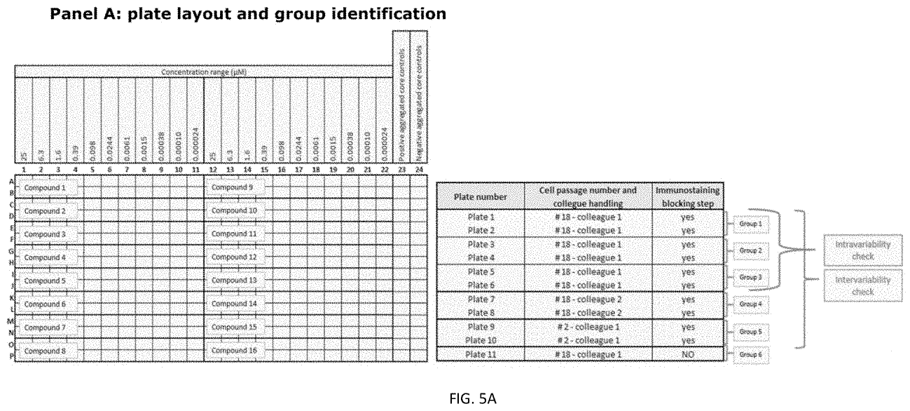

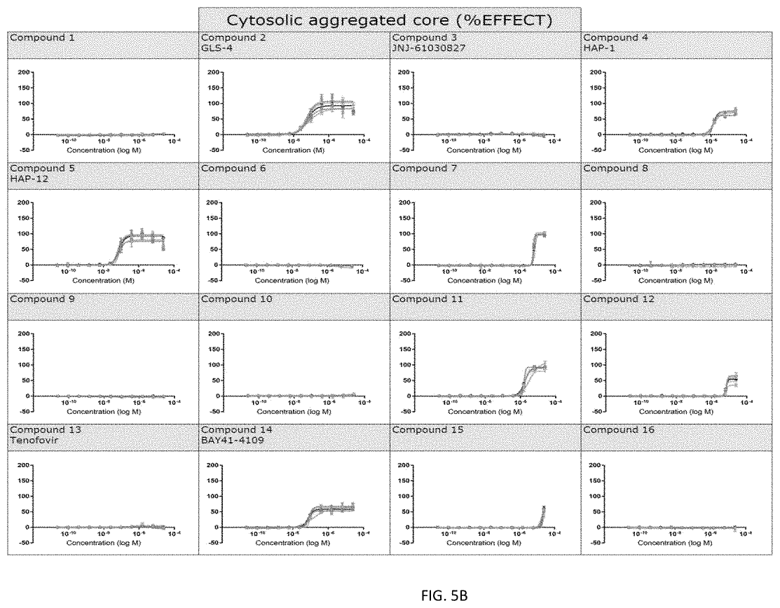

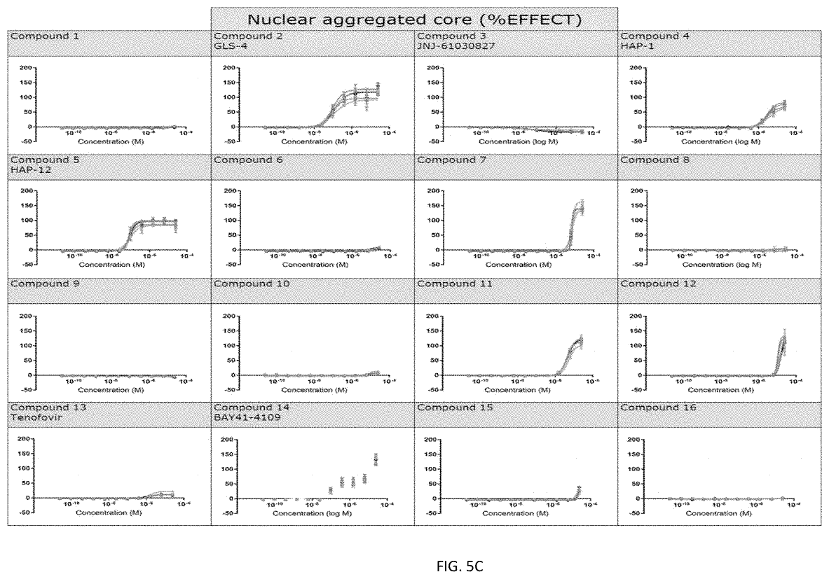

The reproducibility of results using the Abcam monoclonal mouse core antibody and 72-hour incubation was tested by performing an inter- and intra-assay variability test. To this end, a selection of 16 CAM-A and CAM-N compounds was used in three independent experiments to test inter-assay variability, while within one experiment three plate groups, existing of two parallel plates, were used to test intra-assay variability. In addition, one additional compound plate was tested without performing a blocking step in the immunostaining procedure to investigate if the staining procedure could be simplified. Barcoded poly-D-lysine CellCarrier plates containing reference compounds at 11 concentrations in duplicate were used. In order to combine the inter- and intra-assay variability check, HepG2.117 cells with different passage numbers were used. To assess operator-dependent variability, one plate group was processed by a different operator (FIG. 5A).

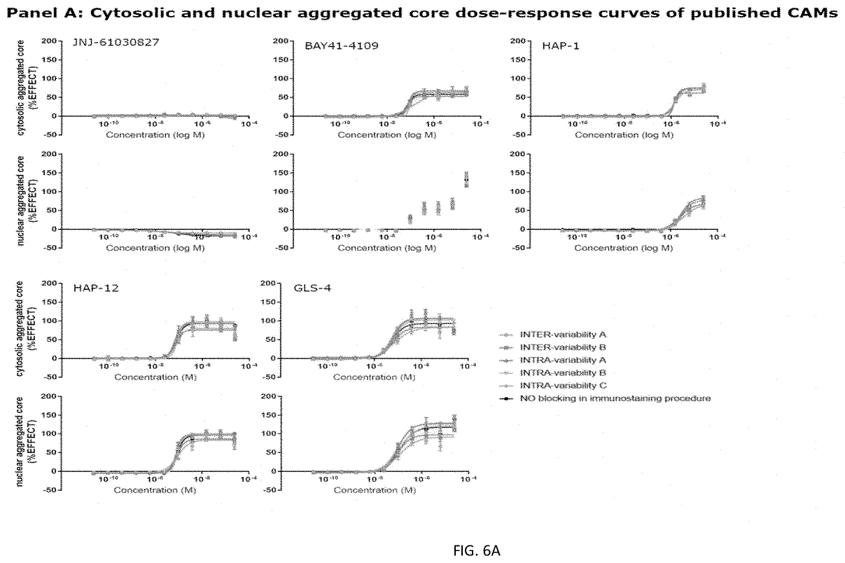

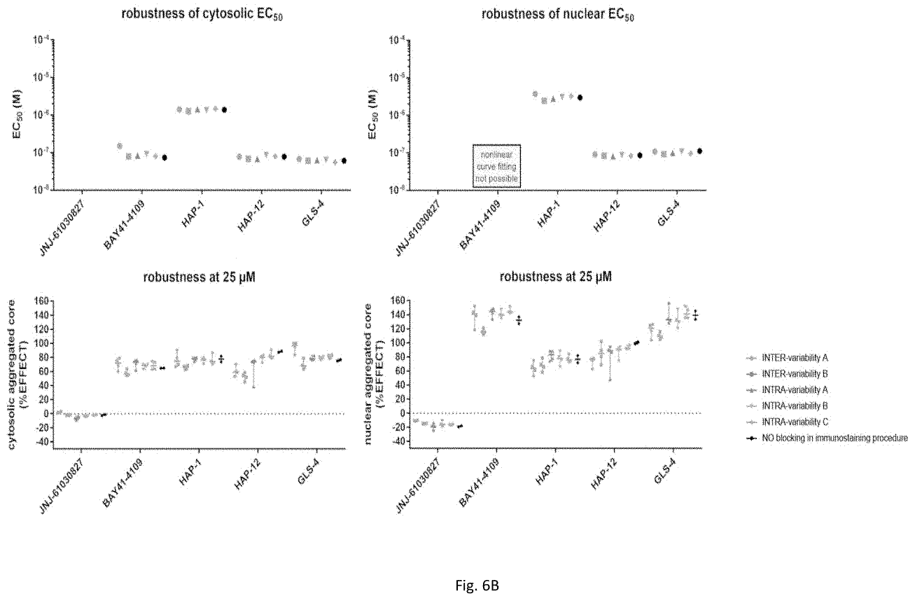

A dose-dependent cytoplasmic and nuclear core speckling phenotype was observed for 8 of the 16 selected compounds, including BAY41-4109, GLS-4, HAP-1, and HAP-12, while in contrast CAM-N compounds (e.g., JNJ-61030827) did not induce any core speckling (representative curves shown in FIG. 6A all curves shown in FIG. 5B). Robustness of the assay is reflected by the narrow range between minimum and maximum calculated EC₅₀ values between the different plate groups for the compounds that induced core speckling (FIG. 6B). The observed range for the minimum and maximum effect observed at the highest tested concentration of 25 μM was slightly broader but is still acceptable for validation of the assay.

Omitting the blocking step in the immunostaining procedure did not affect the quantification of core speckling and therefore this step can be excluded from the protocol (FIG. 6B).

TABLE 2
Robustness of EC50 values and maximal obtained effects

| | Cytosolic aggregated core | | Nuclear aggregated core | |
|---|---|---|---|---|
| | Median EC50 | Median max effect | Median | Median max effect |
| Compound | (μM) | at 25 μM | EC50 (μM) | at 25 μM |
| No./Name | (Range) | (Range) | (Range) | (Range) |
| Compound 11 | 1.78 | 91.7 | 4.44 | 116.2 |
| | (1.64-3.51) | (79.6-104.6) | (4.05-5.19) | (98.7-124.3) |
| Compound 12 | 6.81 | 56.1 | 12.9 | 114.9 |
| | (6.59-6.92) | (35.2-65-5) | (10.4-15.9) | (80.5-132.1) |
| Compound 14 | 0.082 | 68.1 | Nonlinear curve | 139.2 |
| BAY41-4109 | (0.074-0.15) | (57.7-71.2) | fitting not possible | (115.9-145.5) |
| Compound 15 | 20.6 | 56.2 | 22.1 | 30.0 |
| | (18.9-22.2) | (38.6-62.0) | (21.6-40.5) | 19.2-38.1) |
| Compound 2 | 0.062 | 78.8 | 0.10 | 135.1 |
| GLS4 | (0.055-0.068) | (69.5-94.6) | (0.093-0.11) | (109.8-142.4) |
| Compound 4 | 1.39 | 76.5 | 3.04 | 76.9 |
| HAP-1 | (1.28-1.47) | (65.7-77.7) | (2.47-3.72) | (63.8-82.2) |
| Compound 5 | 0.078 | 72.1 | 0.085 | 86.2 |
| HAP-12 | (0.067-0.086) | (52.6-88.1) | (0.080-0.091) | (73.3-100.0) |
| Compound 7 | 5.96 | 97.6 | 6.69 | 138.9 |
| | (5.82-6.84) | (96.0-103.5) | (6.46-7.23) | (129.5-152.5) |

### Assay Effectiveness for Screening Purposes: Z′ Value

Beside reproducibility of the assay, it is desirable for the assay to be effective for screening purposes. Since the SB value may not be the best indicator of assay effectiveness, suitability of a cellular screening assay is often measured by calculating the Z′ factor of the assay. The Z′ factor considers the means and the standard deviations of both the low signal controls and high signal controls. The Z′ value represents an effective assay if between 0.5 and 1.0. A complete 384-well plate was analyzed of which 160 wells contained core-expressing HepG2.117 cells treated with 1 μM GLS-4 to induce core speckling (high controls), 160 wells contained untreated core-expressing HepG2.117 cells (low controls), and 64 wells contained core suppressed HepG2.117 (background controls), which were used by the image analysis to calculate the core expressing cell classification threshold.

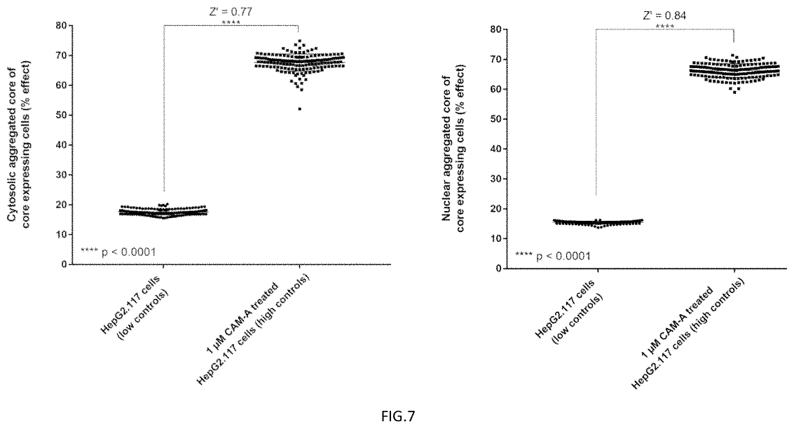

The overall cytosolic and nuclear core speckling of all imaged cells (expressing core or not) was significantly different between the low controls and high controls (p<0.0001, via unpaired t-test) and resulted in Z′ values of 0.76 and 0.80, respectively (FIG. 7). These Z′ values reflect a high-quality assay that can differentiate between positive and negative results.

Because the focus is to test if core proteins aggregate or not, the core suppressed cells are not valuable in the analysis. To exclude them, a re-analysis of the data was performed whereby the background control wells were used in a workflow to calculate a core intensity threshold value that defines the core-expressing subset of the imaged HepG2.117 cells. The Z′ determination for the subset of cells expressing core resulted in comparable cytosolic Z′ values of 0.77, while the nuclear aggregated core Z′ value was improved to 0.84.

With the inter- and intra-assay variability results that indicate robust performance of the assay and an effectiveness that is improved if core speckling is analyzed for the core-expressing HepG2.117 subset of imaged cells, the assay was used for the screening of compounds to identify non-HAP chemical scaffolds able to induce core aggregation.

### Combined Biochemical and High-Content Image-Based Screening Approach to Identify CAMs Inducing Aberrant Core Structures

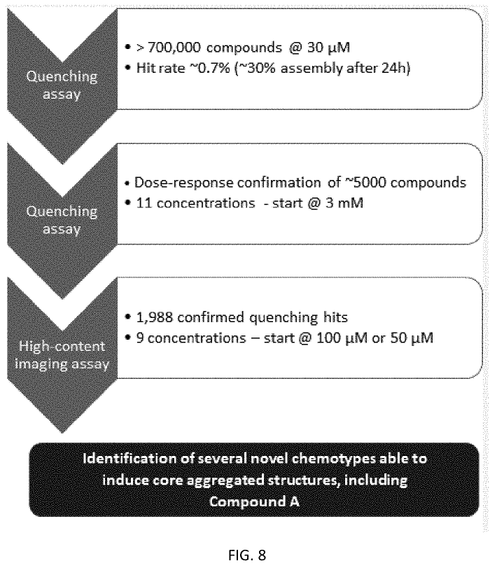

The high-throughput screen for the identification of novel CAM-A chemotypes started with a biochemical fluorescence quenching assay based on the modified recombinant HBV core protein Cp*150 to identify compounds that have an effect on capsid assembly. A flowchart of the screening approach is shown in FIG. 8. More than 700,000 compounds were screened at a fixed concentration of 30 and approximately 5,000 had an effect on assembly (meeting the hit selection criteria of 30% assembly after 24 hours), representing a hit rate of about 0.7%. The identified hits were ordered for an eleven-point dose range secondary confirmation test in the same biochemical quenching assay and led to 1,988 confirmed CAM scaffolds. Since the biochemical assay does not discriminate between CAM-Ns and CAM-As, all 1,988 confirmed hits were profiled in the high-content imaging assay described above in dose-response with a start concentration of 50 μM or 100 μM. In this assay, the induction of core speckling indicates that the test compound has a CAM-A mechanism of action compared to CAM-N compounds that will lead to a more uniform core distribution. The high-content phenotypic analysis for the 1,988 hits of the high-content assay led to the identification of a novel non-HAP CAM-A scaffold (Compound A), able to induce cytosolic and nuclear core aggregation.

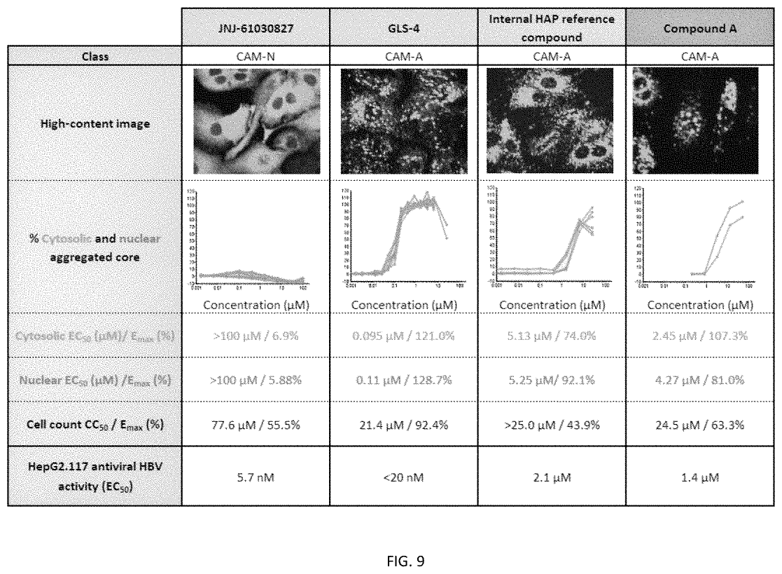

Compound A was found to have a potency to induce cytosolic and nuclear aggregation, with EC₅₀ values of 2.45 μM and 4.27 μM, respectively, which is comparable to the EC₅₀ values of an internal HAP scaffold that shows a cytosolic core speckling EC₅₀ value of 5.13 μM and a nuclear core speckling EC₅₀ of 5.25 μM (FIG. 9). Also, in line with the comparable potency, the maximal observed effect (Eₘₐₓ) was equal for both compounds: 107%/81% for cytosolic and nuclear core speckling for compound A versus 74%/92% for the internal HAP scaffold. The reference compound GLS-4 tested in a full dose range has a higher potency to induce core aggregation since a lower EC₅₀ value of 0.095 μM for cytosolic core speckling and 0.11 μM for nuclear aggregation was observed. Also the maximum effect of core aggregation in the cytoplasm and nucleus was higher for GLS-4 (Eₘₐₓ of 121% and 129%, respectively). Observed Eₘₐₓ values >100% means that there is more core aggregation observed in the HepG2.117 cells compared to the high control samples which are created by 1 μM GLS-4 treatment.

Compound A was further profiled for HBV DNA decline in HepG2.117 cells. In this assay, Compound A had an antiviral EC₅₀ value of 1.4 μM in the absence of cytotoxicity up to a concentration of 50 μM, resulting in a good selectivity index (>35).

### Confirmation of Compound a CAM-A Mode of Action in Capsid Depletion Assay and Electron Microscopy

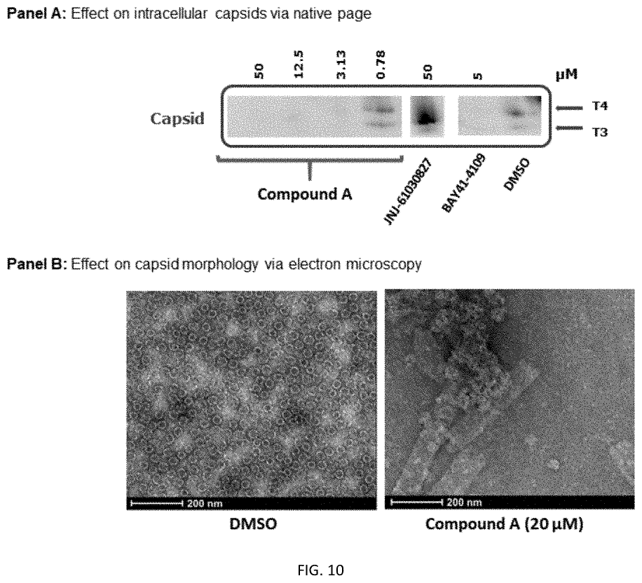

The induction of core aggregation induced by Compound A in the cellular high-content imaging assay indicates that this compound is a CAM that belongs to the CAM-A classification where core proteins are induced to form aggregated structures. To confirm this, HepG2.117 cells were treated for 3 days with compound A or reference compounds, and lysates were used in native polyacrylamide gel electrophoresis (page) followed by western blotting to detect the core protein. A dose-dependent depletion of intracellular capsids was observed after treatment with compound A. This finding was in-line with results generated with the reference CAM-A compound BAY41-4109. In contrast, no capsid depletion was observed after treatment with CAM-N compound JNJ-61030827 (FIG. 10, panel A).

Electron microscopy studies with recombinant HBV core protein incubated with Compound A led to the formation of aberrant core structures whereas DMSO treatment did not affect the formation of typical icosahedral HBV core protein capsids (FIG. 10, panel B). Therefore, it can be concluded that Compound A can be classified as a novel CAM-A scaffold.

1. A method of identifying a compound that induces the formation of Hepatitis B Virus (HBV) aberrant viral capsid structures, the method comprising the steps of:
a) providing a plurality of HBV-replicating cells;
b) contacting the HBV-replicating cells with at least one test compound;
c) fixating the HBV cells of step b);
d) incubating the fixated HBV cells of step c) with an antibody that specifically binds to HBV core protein, thereby labelling HBV core protein fixated HBV cells;
e) imaging the HBV core protein-labeled fixated HBV cells; and
f) determining the presence of an HBV core protein speckle phenotype,
wherein the presence of an HBV core protein speckle phenotype indicates that the test compound induces the formation of HBV aberrant viral capsid structures.

2. The method of claim 1, wherein the HBV-replicating cells inducibly express HBV pregenomic RNA (pgRNA), thereby producing HBV.

3. The method of claim 2, wherein HBV pgRNA expression is induced with a tetracycline-inducible promoter.

4. The method of claim 1, wherein the HBV-replicating cells are liver-derived cells.

5. The method of claim 4, wherein the liver-derived cells are selected from the group consisting of HepG2, HepaRG, Huh7, primary hepatocytes, primary human hepatocytes, and Tupaia belangeri hepatocytes.

6. (canceled)

7. (canceled)

8. The method of claim 1, wherein the test compound is a heteroaryldihydropyrimidine (HAP) or derivative thereof; or a non-HAP derivative.

9. The method of claim 1, wherein the antibody that specifically binds to HBV core protein is polyclonal or monoclonal.

10. The method of claim 1, wherein the antibody that specifically binds to HBV core protein is labeled with a detectable moiety.

11. The method of claim 1, wherein the HBV core protein in fixated HBV-cells are further incubated with a fluorescent label detection antibody that specifically binds to the HBV core protein antibody prior to imaging step d).

12. (canceled)

13. The method of claim 1, wherein the imaging is performed by confocal microscopy.

14. The method of claim 1, wherein the HBV-replicating cells or HBV core protein-labeled HBV-replicating cells are further incubated with a nuclear dye and cell demarcation dye.

15. (canceled)

16. (canceled)

17. The method of claim 1, wherein the HBV-replicating cells are incubated with the at least one test compound for about 12 hours to about 120 hours.

18. The method of claim 1, wherein the HBV-replicating cells are incubated with the at least one test compound for about 72 hours.

19. The method of claim 1, wherein the HBV-replicating cells are fixed and permeabilized following incubation with the at least one test compound.

20. (canceled)

21. The method of claim 1, wherein determining the presence of the HBV core protein speckle phenotype comprises the steps of:
i) quantifying the number and/or image intensity of HBV core protein aggregates in the cell nucleus and cell cytoplasm of individual HBV-replicating cells within the plurality of HBV-replicating cells;
ii) calculating the average number and/or image intensity of HBV core protein aggregates in the cell nucleus and cell cytoplasm, thereby producing an average value; and
iii) comparing the average value of HBV core protein aggregates to an average value of HBV core protein aggregates in HBV-replicating cells that were not incubated with the at least one test compound,
wherein a higher average value in HBV-replicating cells incubated with the at least one test compound compared to the HBV-replicating cells that were not incubated with the at least one test compound indicates the presence of the HBV core protein speckle phenotype.

22. The method of claim 21, wherein determining the presence of the HBV aggregated core protein phenotype comprises the steps of:
i) quantifying the levels of HBV core protein by calculating the average core staining intensity of individual HBV-replicating cells
ii) classifying each HBV-replicating cell as core-expressing or core-suppressed by comparing the intensity calculated in i) to an intensity threshold calculated based on core-suppressed cells (“background control”)
iii) quantifying the extent of HBV core protein aggregation in each cell by calculating a set of texture features derived from the Core staining channel; selecting the texture feature that results in the best separation between negative control wells (core-expressing cells but no compound treatment) and positive control wells (core-expressing cells treated with a reference compound known to induce Core protein aggregation); thereby producing an average value of the selected texture feature across all cells or only the cells classified as core-expressing in step ii)
iv) normalizing the selected texture feature such that the median of the negative control wells on the plate corresponds to a value of 0% and the median of the positive control wells on the plate corresponds to a value of 100%.

23. The method of claim 1, wherein a compound that does not induce the formation of HBV aberrant viral capsid structures produces an HBV core protein uniform imaging phenotype.

24. The method of claim 23, wherein the HBV core protein uniform imaging phenotype comprises an even distribution of HBV core protein in the nucleus and cytoplasm of the HBV-replicating cells.

25. The method of claim 1, wherein the HBV aggregated core protein phenotype comprises an aggregated dot pattern in the nucleus and cytoplasm of the HBV-replicating cells.
